SEQUENCE LISTING (2 sequences)

SEQ ID NO: 1 - protein, 759 aa, Artificial Sequence
  NOVEL CHERKASKY FUSION PROTEINS CONTAINING ANTIBODY BINDING PROTEINS OR THE REGIONS THEREOF
MGILSFLPVLATESDWADCKSPQPWGHMLLWTAVLFLAPVAGTPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRIMSIHFSSPVFTSRSAAFSGRGAQVRLSSARPGGLGSSSLYGLGASRPRVAVRSAYGGPVGAGIREVTINQSLLAPLRLDADPSLQRVRQEESEQIKTLNNKFASFIDKVRFLEQQNKLLETKWTLLQEQKSAKSSRLPDIFEAQIAGLRGQLEALQVDGGRLEAELRSMQDVVEDFKNKYEDEINHRTAAENEFVVLKKDVDAAYMSKVELEAKVDALNDEINFLRTLNETELTELQSQISDTSVVLSMDNSRSLDLDGIIAEVKAQYEEMAKCSRAEAEAWYQTKFETLQAQAGKHGDDLRNTRNEISEMNRAIQRLQAEIDNIKNQRAKLEAAIAEAEERGELALKDARAKQEELEAALQRGKQDMARQLREYQELMSVKLALDIEIATYRKLLEGEESRLAGDGVGAVNISVMNSTGGSSSGGGIGLTLGGTMGSNALSFSSSAGPGLLKAYSIRTASASRRSARD

SEQ ID NO: 2 - protein, 472 aa, Artificial Sequence
  NOVEL CHERKASKY FUSION PROTEINS CONTAINING ANTIBODY BINDING PROTEINS OR THE REGIONS THEREOF
MGILSFLPVLATESDWADCKSPQPWGHMLLWTAVLFLAPVAGTPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRILTRELCCCHTITQRPIFIVVVGEGNGKYELVGIRDQQRQQDEQEEEPEEVRRYSARLSEGDIFVIPAGYPISVNASSNLRLIGFGINAYENQRNFLAGSEDNVIRQLDREVKELTFPGFAEDIERLIKNQQQSYFANAQPQQQQQQQQQQQQQQQQQQQQQSEKEGRRGRRGPISSILSTLY

(19) United States
(12) Patent Application Publication
(10) Pub. No.: US 2008/0200652 A1
(43) Pub. Date: Aug. 21, 2008

(54) Novel Cherkasky fusion proteins containing antibody binding proteins or the regions thereof

(76) Inventor: Alexander Cherkasky, Duesseldorf (DE)

Correspondence Address: Alexander Cherkasky, Prinz-Georg Str. 5, Duesseldorf, 40477, DE

(21) Appl. No.: 12/003,272

(22) Filed: Dec. 21, 2007

(30) Foreign Application Priority Data
Jun. 20, 2005 (DE) 10 2005 028 619.4
Jun. 21, 2005 (DE) 10 2005 028 545.7
Jun. 7, 2006 (IB) PCT/IB2006/001490

(51) Int. Cl. C07K 16/00; C07H 21/02; C12N 15/66; C07D 207/04

(52) U.S. Cl. 530/387.3; 536/23.1; 435/320.1; 548/572

(57) ABSTRACT

Disclosed are novel fusion proteins containing antibody binding regions and certain other regions. Said fusion proteins can form complexes with antibodies, which can thus be modified and be arranged three-dimensionally. Furthermore, the effect thereof can be boosted.

### BACKGROUND OF THE INVENTION

The invention relates to the fields of molecular biology immunology and biotechnology.

From the level of technology are known works and publications (DE 102 02 191 A1, DE 103 50 131 A1 and WO 2005/040382) and they describe fusion proteins containing both Fc region binding domains, such as for example either Staphylococcal protein A (SPA) or Fc Receptor Ecto or Ligand-binding-domaine and sequence specific nucleic acid binding domains, as well as fusion proteins, containing both antibody binding regions such as preferably Staphylococcal protein A (SPA), an extracellular region of the Fc Receptor CD 64 or their regions and microtubule binding regions, preferably gephyrin, Tau, MAP or their regions.

These compounds can not be used for many various different purposes.

The broad spectrum as well as uses or utilities of the fusion proteins containing antibody binding regions are still not evolved.

### SUMMARY OF THE INVENTION

The object of the invention lies in novel compounds with novel features and utilities or uses, wherein these compounds can bind, arrange antibodies and (or) enhance them in their action against their antigens.

The object of the invention will be solved through fusion proteins containing antibody binding regions according to the present invention.

The fusion proteins containing antibody binding proteins or their regions comprise novel classes of compounds, materials, drugs or medications, diagnostic, sensor, test or control systems, adhesives, dyes and labelling substances as well as classes of many other substances And systems.

### DESCRIPTION OF THE INVENTION

On certain examples this broadness should be presented or these numerous uses, utilities and advantages should be presented. These numerous uses and advantages are not limited to the examples listed below.

### Example 1

### Cherkasky Fusion Proteins Containing IgE-Antibody Binding Regions and Hydrophil Regions

Increased levels of IgE-antibodies indicate and are typically for asthma, atopic eczema, certain allergies and inflammations. Therefore the goal and purpose of certain therapies are directed to the decrease of levels of IgE antibodies; thus it means, to develop drugs, that remove IgE molecules and thus decrease their levels. Known are Anti IgE antibodies developed by Genentech and traded under the trade mark Xolair.

Because the removal of IgE antibodies by using other (IgE) antibodies occurs through macrophages i.e. through activation of macrophages, which release inflammatory mediators, the action of anti-IgE-antibodies is not so efficient as anticipated. Cherkasky Fusion Proteins containing both IgE antibody binding regions and hydrophil regions can bind IgE antibodies and remove them by hydrophil regions through circulation from the area of inflammation and thereby these fusion proteins do not depend from the activation of macrophages.

Thereby, the treatment can be carried out more effectively with reduced side effects.

### Example 2

### Cherkasky Fusion Proteins Containing IgE-Antibody Binding Regions and Receptors

The purpose was described in the Example 1.

The receptors of these fusion proteins bind preferably inflammation factors such as TNF-alpha, C5a, C3a and C4a. These fusion proteins possess therefore a double action mechanism. On the one side, the binding of IgE antibodies to their receptors will be blocked, prohibited because of the binding fusion proteins according to the present invention, i.e. to the Example 2, on the other side, the released inflammatory factors, such as TNF Alpha will be bound to the receptor regions of the fusion proteins.

This inflammation-inhibiting double action can allow the more effective treatment with reduced side effects.

### Example 3

### Cherkasky Fusion Proteins Containing Both Either IgE- or IgG Antibody Binding Regions and Enzymes

Enzymes, preferably DNAases, DNAse 1, S1 endonuclease, catalase or peroxidase, act also as inhibitors of inflammations as well as they alleviate by the Systemic Lupus Erythemathosus (SLE).

This anti-inflammatory double action of fusion proteins according to this example is similar with the double action described in the Example 2.

### Example 4

### Cherkasky Fusion Proteins Containing Both Either IgE or IgG-Antibody Binding Regions and Receptors and Enzymes

These fusion proteins possess the triple anti-inflammatory action of the fusion proteins, that were described in examples 2 and 3, whereas the enzymes are DNAse 1, 51 endonuclease, catalase, peroxidase or other anti-inflammatory enzymes and receptors are for example TNF Alpha receptor, receptors C5a, C3a and C4a or other receptors, that bind inflammation mediators.

### Example 5

### Cherkasky Fusion Proteins Containing Antibody Binding Regions and Fluorescent Proteins or their Regions

Through these fusion proteins, antibodies, such as IgG or IgE, can be labelled specifically. The main advantage of the labelling of antibodies through these fusion proteins is the precise labelling.

As an example for a fluorescent protein, the GFP (green fluorescent protein) or its region can be used.

### Example 6

### Cherkasky Fusion Proteins Containing Antibody Binding Regions and Metal Binding Proteins or their Regions

These fusion proteins, which are complexed with metal atoms or ions, can be directed or manipulated and orientated through magnetic fields. Therethrough will be solved the object of molecular orientation. The metal binding proteins are for example, astacin (from fishes Astacus), forritin, cooruloplasmin, a cystein-rich region and many other proteins or their regions.

### Example 7

### Cherkasky Fusion Proteins Containing Antibody Binding Regions, Metal Binding Regions and Fluorescent Regions

These fusion proteins can label and can be orientated, thus this is an advantage for example by the isolation and purification of certain antigens, which are bound to antibodies, that are bound to fusion proteins.

### Example 8

### Cherkasky Fusion Proteins Containing Antibody-Binding-, Polymer-Binding and Metal-Binding Regions

These fusion proteins can bind a polymer, through a polymer binding region, preferably a biopolymer such as for example a DNA sequence or a polysaccharide such as cellulose, chitin, pectin, starch; through antibody-binding regions they can bind, for example IgG or monoclonal antibodies and through metal-binding regions, which are complexed with metal ions or atoms, they (these fusion proteins) can be directed and orientated.

Such complexes consisting from fusion proteins and polysaccharides can be used, for example, for infiltration of certain substances, preferably antigens, such as viral antigens.

### Example 9

### Cherkasky Fusion Proteins Containing Antibody-Binding, Polymer-Binding and Fluorescent Proteins or their Regions

The advantages and use, utilities of these fusion proteins are similar with those of the example 8, whereby the fluorescent regions change their color, brightness or intensity in dependence from concentrations of the bound or infiltrotcd antigens.

### Example 10

### Cherkasky Fusion Proteins Containing Antibody-Binding-, Metal-Binding, Polymere-Binding and Fluorescent Proteins or their Regions

The uses of these fusion proteins are similar with those, of the examples 8 and 9; thereby the better molecular orientation by using magnetic fields will be achieved through metals, that are bound or complexes on the metal binding regions.

### Example 11

### Cherkasky Fusion Proteins Containing Antibody Binding Region and at Least One Receptor or it Region

The antibody binding region is preferably a IgG binding region, such as for example SPA, whereas the receptor binds for example an inflammation mediator such as TNF Alpha, C5a, C4a or C3a.

The action is alleviating by SLE and similar with the description in the examples 2-4.

### Example 12

### Cherkasky Fusion Proteins Containing Antibody Binding Region and Ligands or their Regions

Ligands are, for example, C3b or C4b proteins of the immune system or their regions, that bind CR1 receptors of erythrocytes and are participated on the opsonization (opsonation) and removal of immune complexes. Through these fusion proteins, antibodies, such as IgE by inflammations or IgG autoantibodies for example by Epidermolysis bullosa acquesita (EBA), will be bound and removed and degraded by an alternative way, thus it allows to achieve alleviation.

### Example 13

### Cherkasky Fusion Proteins Containing Antibody-Binding Regions, Receptors and Ligands

Receptors are, for example, receptors for inflammatory factors, such as TNF Alpha; ligands are, for example C3b and C4b or their regions. These fusions have a double anti-inflammatory action, such as proteins, that are described in the examples 2-4, 11 and 12.

### Example 14

### Cherkasky Fusion Proteins Containing Antibody Binding Regions, Receptors and Enzymes

Receptors are for example receptors for inflammation factors and enzymes are for example DNAse 1, S1 endonuclease, catalase or peroxidase and they act anti-inflammatory.

The additional binding the antibodies IgE or IgG increases the anti-inflammatory action of the fusion proteins according to this example.

### Example 15

### Cherkasky Fusion Proteins Containing Antibody-Binding Regions or their Regions and Autoantigens or their Regions

These fusion proteins can be used for diagnosis of autoimmune diseases. The fusion proteins will be added to the serum.

If autoantibodies are available in the serum in small or bigger concentrations, so these autoantibodies bind autoantigenes or autoantigenic regions of the fusion proteins. The Fc regions of the antibodies will be bound. Thus, the multimolecular complex will be built, that precipitates.

If there are no or very few autoantibodies in the serum, thus the autoantigenic regions of the fusion proteins will not be bound and thus the multimolecular complex will not arise. The precipitation can not be therefore observed in this case.

### Example 16

### Cherkasky Fusion Proteins Containing Antibody-Binding-, Autoantigenic and Fluorescent Regions

The use of these fusion proteins as diagnostic tools is similar with the use in the example 15, whereas the arising of complexes will be better visualized and these complexes will be made more clear.

### Example 17

### Cherkasky Fusion Proteins Comprising Antibody-Binding and T Cell Reaction Triggering Regions

The modification of monoclonal IgG-antibodies with these fusion proteins will lead to the improved use of antibodies against viral, fungal, bacterial and protozoal disease causing agents because these disease causing agents will be attacked by T cells.

### Example 18

### Cherkasky Fusion Proteins Comprising Antibody-Binding Regions or their Regions and Storage Proteins or their Regions

Storage proteins can be selected for example either from animal proteins such as caseins or from plant proteins such as vicilins, legumins, gliadins, glutenins, lupins, avenins, secalins, hordeins, trifoliin. They can be used as fusion parts. The use of these fusion proteins or fusion proteins-antibody complexes can be in the creation of novel materials, that can be either amorphous or elastic, and can recognize and bind certain antigens, such as preferably those located on the surface. Such materials can be used preferably as novel antigen-specific filters, as well as security signs or marks that can be upstroked on certain surfaces.

### Example 19

### Cherkasky Fusion Proteins Comprising Antibody-Binding Proteins or their Regions and Structural Proteins or their Regions

The structural proteins can be selected for example from the following proteins: lamprin, a cell protein, resilin, abducin, crystallins alpha, beta and gamma, collagen and keratin.

The uses are similar with those of the example 18, whereas the resulting material can be either elastic or solid.

### Example 20

### Cherkasky Fusion Proteins Comprising Antibody-Binding Proteins or their Regions as Well as Storage Proteins or their Regions and Structural Proteins or their Regions

The materials composed from these fusion proteins or fusion protein-antibody complexes have similar used and features, as those described in the examples 18 and 19.

### Example 21

### Cherkasky Fusion Proteins Comprising Antibody Binding Proteins or their Regions, Metal Binding Regions and Storage Proteins or their Regions

The features and uses are similar with those that were described in the example 18-20.

### Example 22

### Cherkasky Fusion Proteins Comprising Antibody Binding Proteins or their Regions, Metal Binding Regions and Structural Proteins or their Regions

The features and uses are similar with those described in the examples 18 and 19.

### Example 23

### Cherkasky Fusion Proteins Comprising Antibody Binding Proteins or their Regions, Metal Binding Regions or their Regions, Structural Proteins or their Regions and Storage Proteins or their Regions

The features and uses are similar with those described in the examples 18 and 19. Further examples comprise fusion proteins comprising antibody binding proteins or their regions and plant proteins such as for example Phloem Protein 1 (PP1) from Cucurbitas, fish proteins such as for example astacin from Astacus, neuroproteins such as for example GFAP (Glial Fibrillary Acidic Protein), MBP (Myelin Basis Protein), a polymer binding protein-enzyme fusion protein, whereas the polymer binding region is preferably a cellulose binding region such as for example Cip A; a thermostable protein rubredoxin, antifreeze protein or glycoprotein, a serine or serine rich region, a laminin, actin, myosin, a cytokin, an interleukin, a blood protein, such as e.g. serum albumin, a transprotein or a transport protein, a basic protein or its region, an acidic protein or its region, a spacer, a hydrophobic protein or its region a hydrophil protein or its region, a cell protein, a cell nuclear protein or their regions, a neurofilament or its region, a viral protein such as for example a phage T4 protein, Tobacco Mosaic Virus TMV-protein, an opsonin, CRP or C-reactive protein, MBP or Mannose Binding protein, nucleic acid binding proteins or their regions, LH, FSH, vicilin like protein or any other protein or it regions.

The fusion proteins or fusion protein-antibody-complexes can be used as novel materials for various purposes. All of them have either elastic or amorphous or solid mechanic features and a specific binding affinity to antibodies, that are bound on surfaces, as well as can recognize specific antigens and thus can be used as a basis for construction of sensors. As soon as these fusion protein-antibody-complexes conduct electrical current and these antibodies recognize specifically their antigens, the conducting features and resistance will be changed in a small manner, but this alteration can be easily registered and measured. This is the basis for the construction of novel diagnostic-, test-, control-, sensor and alarm or warning systems that can be reduced or minimized and can work more efficiently in comparison with current electronic systems with immobilized antibodies, because through-big concentration or condensation of fusion proteins or fusion protein-antibody complexes, already few antigens or very small concentrations of antigens are enough for triggering specific signals for registration or measuring.

These few antigens, for example dangerous toxines, such as Anthrax, Botulinus, or Tetanus-toxins, viruses such as HIV or Ebola virus, bacteria such as Bacillus anthracis are enough for the registration through interactions with bound antibodies. The bound antibodies or fusion protein-antibody complexes build, form or shape an amorphous or an elastic mass, that can contain salts and that represents a sensor compound of the registration devices according to this invention. Electrical current will be conducted through this mass and each interaction of antigens with the bound antibodies of the fusion proteins-antibody-complexes changes or alterates the current strength and the resistance.

These (antigene and affinity specific) changes can be registered and analysed or interpreted, and this leads to a clear result.

### DESCRIPTION OF THE DRAWINGS

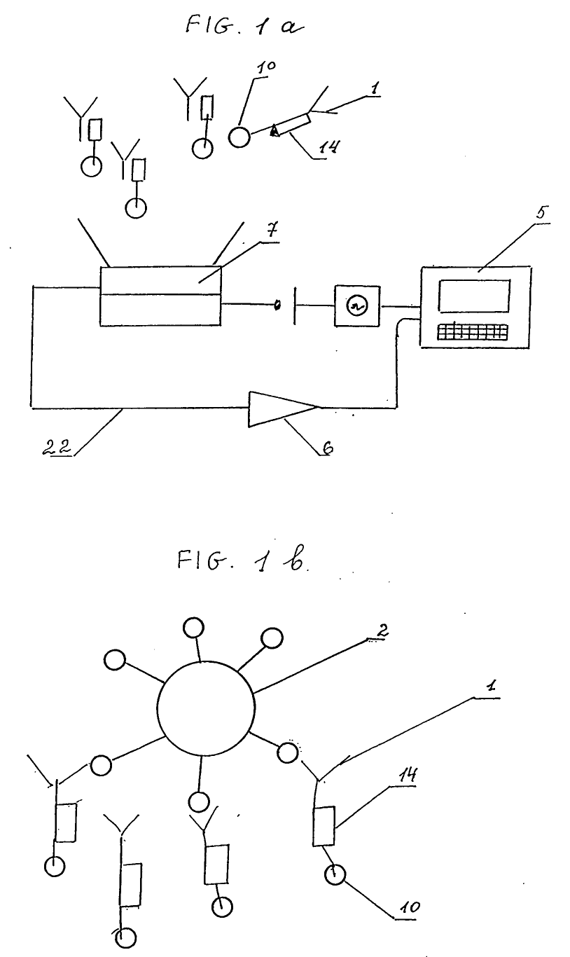

In the FIG. 1a is schematically represented a system for carrying out of diagnosises controls and tests for registration of certain antigens such as for example HIV-viruses, bacteria, proteins, protozoa, or other molecules, microorganisms or cells or substances.

This system contains the registration body 7, that is a mass consisting from antibody-fusion protein complexes and this mass represents a layer on a surface.

The registration body 7 contains antibodies 1, which are bound to the fusion proteins comprising antibody-binding domains 14 and storage proteins 10, or are bound to structural or other proteins. The registration body 7 is bound through the wire 22 with a current amplifier 6 and an interpretation system or computer 5 for the interpretation of changes of electrical signals.

In the FIG. 1b is schematically shown the interaction of the antigen 2, which is for example a HIV-virus, with antibodies 1. These antibodies are bound to the fusion proteins comprising antibody binding regions 14 and storage proteins 10.

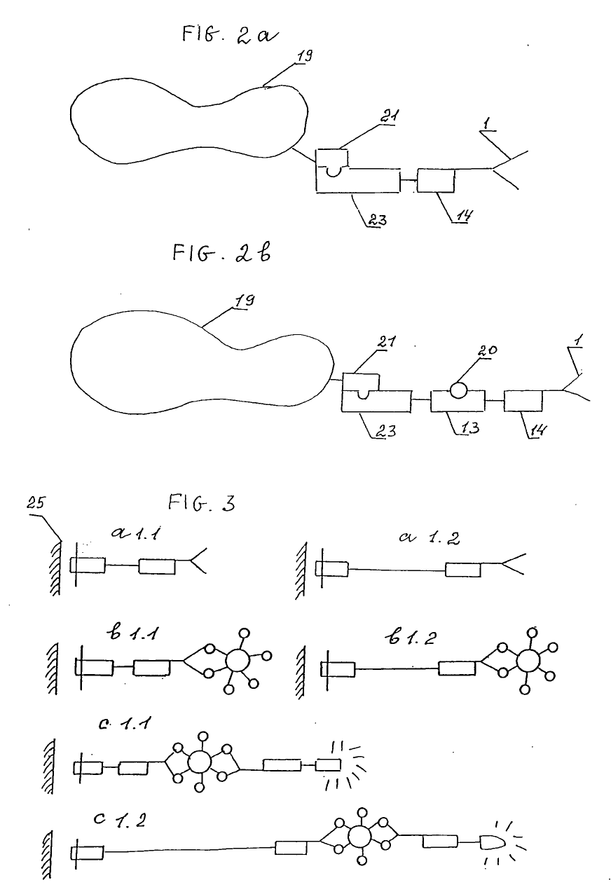

In the FIG. 2a is a fusion protein-antibody complex schematically shown, and this complex binds the CR1 receptor 21 of the erythrocyte 19. The fusion protein-antibody-complex consists both from fusion protein comprising antibody binding domaine 14 and the specific ligand 23, which is C3b or C4b and antibodies 1. Through this fusion-protein, an antigen can be removed and degraded by an alternative way, there by no inflammation will be caused.

The fusion protein-antibody 1-ligand 20-complex in the FIG. 2b binds erythrocytes 19 and contains additional middle receptor domaine 13, that binds e.g. inflammatory mediators such as TNF Alpha and remove them, i.e. thus these inflammatory mediators will be removed from the inflammation site. Thus the additional anti-inflammatory effect will be reached.

In the FIG. 3 is a possible realization of the test-, diagnosis or control system according to the present invention as well as the according molecular process schematically shown.

The antibody, for example a monoclonal antibody in the FIG. 3a 1.1 is bound to a nucleic acid sequence or to a polysaccharide through a fusion protein containing an antibody binding region and a polymere binding region. An antigen, such as for example a virus will be bound by this antibody in the FIG. 3b 1.1. The optical visualization (FIG. 3c 1.1) occurs through the fluorescent protein containing an antibody binding domaine for example SPA and a fluorescent domaine, for example GFP.

The fusion proteins in the FIGS. 3a 1.2, b1.2 and c1.2 contain long spacer regions, for example polyplycine, for the increase of reach.

In the FIG. 3a2, b2 and c2 is schematically shown the according test process for manual or automatic carrying out. The pin or peg 25 with the grip or grasp 24 contains a molecular surface structure, that accords to the FIGS. a 1.1 or a 1.2. This pin in the FIG. 3a3 will be added into the vessel 26 with a blood probe or serum, then the pin will be kept for example for 2 minutes in the vessel and after this removed. Than, this pin (b2) will be added to the solution containing fusion protein-antibody complexes. These fusion proteins consist from antibody binding regions and fluorescent regions.

If antigens such as viruses such as for example HIV or SARS or Hepatitis are available in the blood or serum probe, they will be recognized and bound through specific according antibodies of the fusion protein-antibody complexes. Thus, after this pin will be removed from the probe, it will be luminescent. For testing, if this luminescence is based either on high or low affinity of specific or unspecific interactions between antigens and antibodies, the pin will be added to water or to a buffer solution and moved slightly. Than it will be removed from the solution (c2). If the luminescence remains, the intensity will be measured. The luminescence will be interpreted as follows: antigens bound themselves to antibodies of the fusion protein (complexes), and these are bound to polymers, which are bound to a surface of the pin. The fusion protein-antibody-complexes are bound to these antigens to their free sites.

The fusion proteins are bound with their antibody binding domains to antibodies and they emit light through fluorescent domains. Thus, the during luminescence means the fact, that antigens are available in the blood or serum probe. If the luminescence will disappear (or reduce) after slightly move, it means, that there no antigens were available in the serum or in the blood probe.

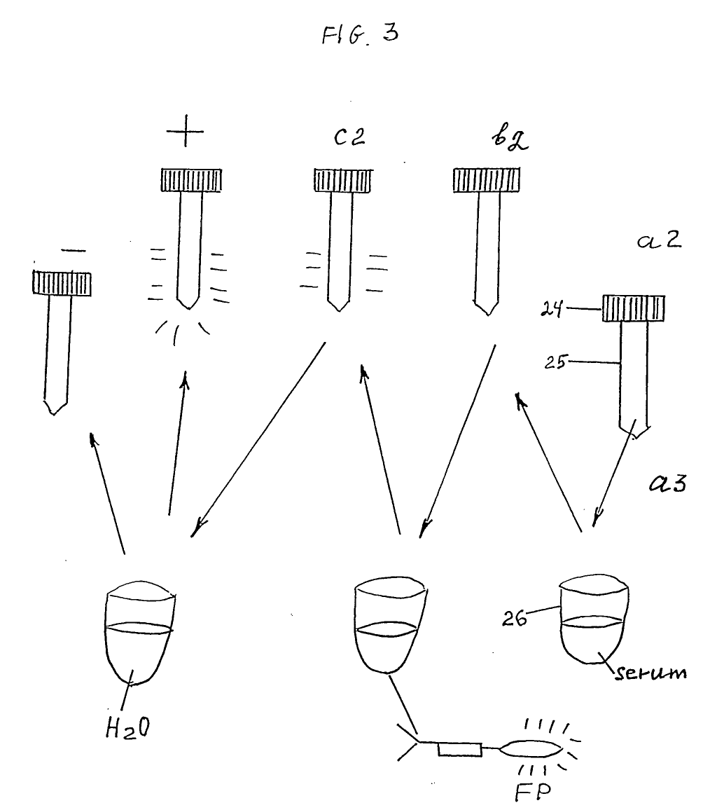

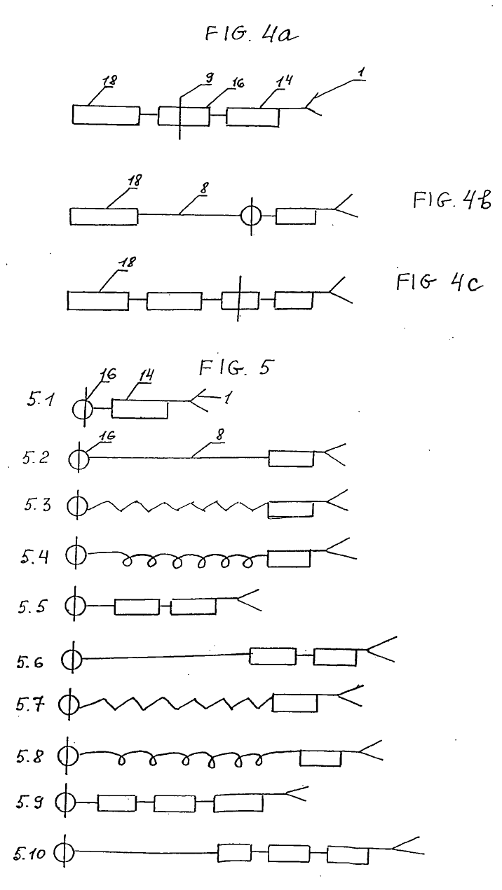

The fusion proteins in the FIGS. 4a, 4b and 4c contain fluorescent regions and can be used as compounds of sensors, because through antigen-antibody-interactions light intensity will be changed. These changes will be registered.

The fusion protein in the FIG. 4a contains a fluorescent region 18, a polymere binding region 16 and the antibody binding region 14, that is bound with an antibody.

The fusion protein interacts with the polymer 9 through the polymere binding region The fusion protein in the FIG. 4b contains additionally the long spacer region 8, such as for example a polyglycine domaine for increased reach.

The fusion protein in the FIG. 4c contains an additionally domaine of a storage protein, structural or a metal protein for increased stability.

In the FIG. 5 are possible constructions of fusion protein-antibody-polymere complexes schematically shown.

The fusion protein in the FIG. 5.1 contains a polymere binding region 16 as well as the antibody binding region 14, that is bound with the antibody 1.

The fusion protein in the FIG. 5.2 contains the additional spacer region 8.

The spacer region of the fusion protein in the FIG. 5.3 contains knicks, that arise through prolines in the polyglycine region.

The spacer region of the fusion protein in the FIG. 5.4 contains a helix. The fusion protein in the FIG. 5.5 contains an additional region of a structural, storage or a metal binding protein for increased stability.

This fusion protein can contain either a linear (FIG. 5.6), knicked (FIG. 5.7) or a helical (FIG. 5.8) spacer region.

The fusion protein in the FIG. 5.9 contains two additional regions, for example a region from a structural protein as well as a region either from a storage or a metal protein.

This fusion protein can contain either a linear (FIG. 5.10), knicked (FIG. 5.11) or a helical (FIG. 5.12) spacer region.

The fusion protein in the FIG. 5.13 contains a terminal domain such as for example GFP or any metal binding domaine, whereas the middle domaine is the polymere-binding domaine.

This fusion protein can contain either a linear (FIG. 5.14), knicked (FIG. 5.15) or a helical (FIG. 5.16) spacer domain.

The fusion protein in the FIG. 5.17 contains two terminal domains either before or after the polymere binding domaine.

The fusion protein can contain either a linear (FIG. 5.18), or knicked (FIG. 5.19) or a helical (FIG. 5.20) spacer region.

The fusion protein in the FIG. 5.21 contains a middle polymere binding region (The definitions “region” and “domaine” are considered to be similar or to be synonyms) and two site domains, whereas the first N- or C-terminal domaine is an antibody binding domaine that is complexed with an antibody. The other domains are for example, a metal binding domaine, the polymere binding domaine, a structural protein domaine and a fluorescent domaine and/or a storage protein domaine for reaching stability, fluorescent and molecular orientation through directing of (with metals) complexed zones.

The knicked domaine can be for example (Pro(Gly)n)m, whereas n and m can be random.

This fusion protein can contain for example either a linear (FIG. 5.22), knicked (FIG. 5.23) or a helical (FIG. 5.24) domaine.

The fusion protein in the FIG. 6 contains an antibody binding domaine 14 and metal binding region 11 and interacts with the antibody 1.

The antibody can be oriented in the solution through both the fusion protein and electromagnetic fields.

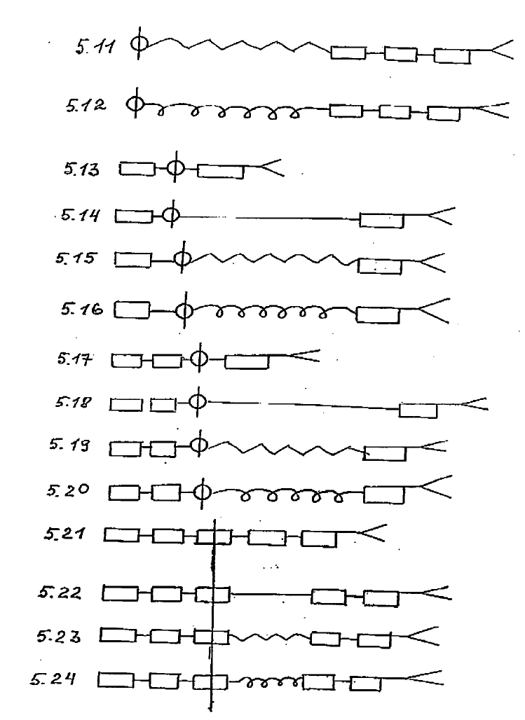

In the FIG. 7 is the complex schematically shown, and this complex contains antibodies and fusion proteins comprising autoantigenes or autoantigenic regions 15 and antibody-binding regions. These fusion proteins serve for diagnosis if certain autoantibodies are available in the blood or serum. If autoantibodies are available, the Fab-fragments of autoantibodies bind autoantogenic regions of the fusion proteins and the complex arises.

If no antibodies are available, accordingly, they do not bind autoantigenic domains of the fusion proteins and the complexes do not arise.

In the FIG. 8 is a polymere-antibody-fusion protein-complex schematically shown. The fusion proteins contain antibody binding domains 14, polymere binding regions 16 and receptors 17 and are bound to the polymere 9 through polymere binding regions 16 and they bind antibodies 1 through the antibody binding regions 14.

The receptors 17 bind ligands 20. The receptors bind for example inflammatory factors such as TNF-Alpha. These are ligands.

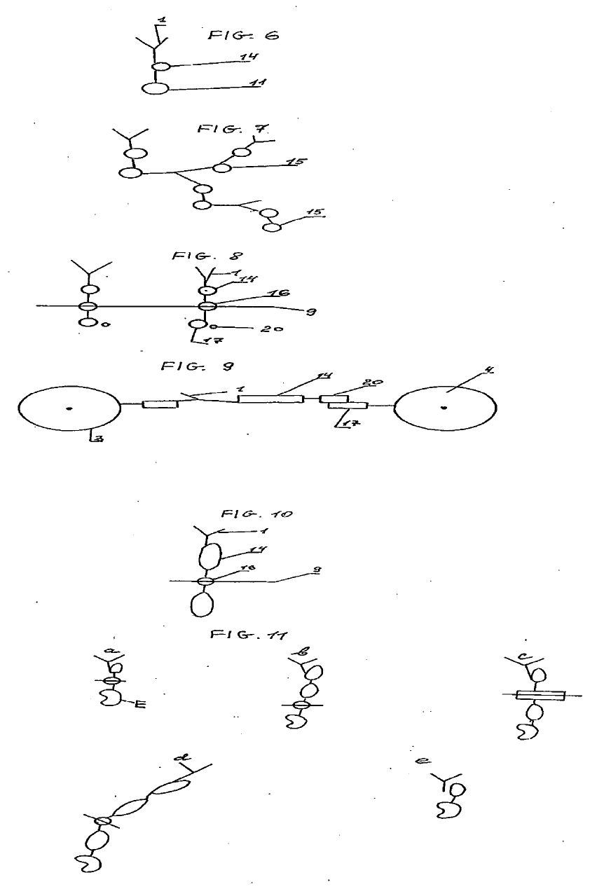

In the FIG. 9 the direction is shown, i.e. the direction or triggering of the T cell 4 reaction against a target cell 3 through fusion protein-antibody complexes according to the present invention. The antibody 1 binds specifically to a target cell specific antigen and the ligand domaine 20 binds the receptors 17 of the T cell.

The ligand domaine is for example a region from HLA-B7.1 or B7.2 and triggers the T cell activation for attacking the target cell.

The fusion protein in the FIG. 10 contains a terminal hydrophil domaine comprising hydrophil amino acids such as serine for increasing the solubility of polymere-fusion protein-antibody-complexes.

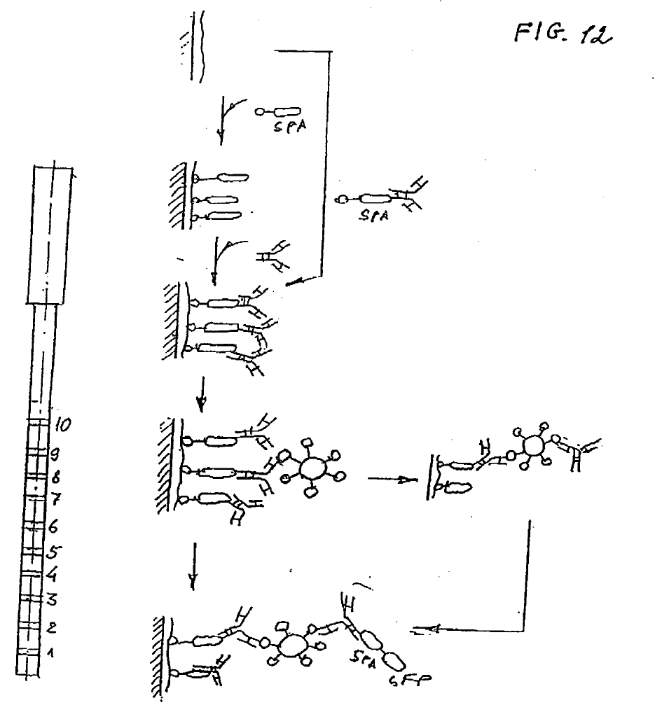

The fusion proteins in the FIGS. 11a, b, c, d and e comprise an enzyme for example protease, nuclease, lipase, catalase, peroxidase or any other enzyme.

In the FIG. 12 is the pin with 10 sectors or segments schematically shown as well as the processes of the preparation and testing are schematically shown. (see FIG. 3). Through the overconcentration of fusion proteins in the solution, the fusion proteins bind themselves to polymers with a high density.

The sequences for proteins are public domaine and they can be obtained from the internet for example by the National Center for Biotechnology Information, NIH, Bethesda, Md., 20894, USA (NCBI http://www.ncbi.nlm.nih.gov).

### EXAMPLE

Cloning and Expression of the Fusion Construct N-Keratin-SPA-C c DNA sequences for keratin and Staphylococcal protein A will be cloned through PCR or reverse transcriptase RT-PCR. The fusion protein will be constructed from different PCR products. The PCR primers will be so constructed, that they contain restriction sites on 5′ and 3′ ends for carrying out further ligation steps. The 5′ and 3′ ends of the keratin-PCR-product contain Bam HI and Hind III restriction sites. The 5′ and 3′ ends of the SPA-PCR product contain Eco RI and Kpn I restriction sites.

Through treatment with Bam HI and Hind III, the keratin PCR product will be ligated into the prepared vector. This vector will be treated with Kpn I and EcoR I for the ligation of the SPA-PCR product into the vector. The ligation product will be transformed, exprimed in E. Coli and than isolated and purified.

In the Seq. 1 is the amino acid sequence of the fusion protein shown, and this fusion protein contains both human low affine receptor for the Fc fragment of the IgG and the human keratin 7.

In the Seq. 2 is the amino acid sequence of the fusion protein shown, and this fusion protein contains both the human low affine receptor for the Fc fragment of the IgG and viciline-like protein from Lupinus albus. The receptor domaine is 290 amino acids long and the length of the viciline-like protein is 182 amino acids. Thus the resulting fusion protein has a length of 472 amino acids.

What is claimed is:

1. Novel fusion proteins, named or entitled Novel Cherkasky Fusion Proteins, comprising at least one antibody binding protein or it region or domaine and at least one non-antibody binding region or a random or any other region.

2. Fusion proteins, named Novel Cherkasky Fusion Proteins, comprising: at least one antibody binding protein or it region, preferably Staphylococcal protein (SPA), any other IgG (Immunoglobulin G) or IgE (Immunoglobulin E) binding region and at least one region selected from the group consisting of the following proteins, fusion proteins or their regions: HLA-B.7.1, HLA-B7.2 or their region, a T cell activating region, an enzyme, DNAse 1, S1 endonuclease, a receptor preferably for inflammatory mediators such as TNF-Alpha. C5a, C3a, C4a a fluorescent protein such as CFP, a ligand, preferably C3b, C4b, an opsonin, CRP or C reactive protein, MSP or mannose binding protein, myelin basic protein, a neural protein, an acidic protein, a basic protein, a hydrophilic protein, a hydrophobic protein, a cystein rich region, an antifreeze protein or glycoprotein, rubredoxin, an autoantigene or autoantigenic region, a structural protein such as preferably resilin, abducin, lamprin, collagen, keratin, a storage protein, casein, a cell protein, a neurofilament, a muscle protein such as preferably myosin or it region, actin, laminin, a cytokine, an interleukin, a blood protein such as preferably serum albumin, a viral protein, a metalloprotein or a metal binding protein, a transport protein or it region, a polysaccharide binding protein (PBD), Cip A colluloso binding region, a nucleic acid binding protein or its region, crystalline, crystallin alpha, beta or gamma, an ichtyoprotein preferably astacin, phloem protein 1 (PP1), phloem protein 2 (PP2) from Cucurbitas, a spacer or linker, a (multi) serine region, a serine rich region, vicilines, legumines, gliadin, glutenin, hordeins, trifoliin, vicilin-like protein from Lupinus albus, a hormone, LH, FSH or PBD-enzyme fusion protein.

3. The fusion proteins of claim 2 wherein they comprise at least one His-tag or any other tag for isolation and purification, a linker, a hinge region or at least one other region.

4. Nucleic Acid and Amino Acid sequences, Vectors, DNA vectors, cloning, expression and transformation systems for the fusion proteins of claim 2 or of claim 3.

5. Use of substances of claim 2 or claim 3 preferably as materials, medications, drugs, dyes, labelling substances, adhesives and as glues and diagnostic, sensor test and control systems.

6. Linker or Spacer comprising the formula (Pro(Gly)n)m, whereas knicks will be built in glycine chains through inserted prolines.

7. Use of polymers for dense arrangement of fusion proteins especially of proteins of claim 2.
